(19) United States
(12) Patent Application Publication
(10) Pub. No.: US 2006/0142731 A1
(43) Pub. Date: Jun. 29, 2006

(54) Floating gastro-intestinal anchor

(76) Inventor: Jeffrey Brooks, Shivtei (IL)

Correspondence Address: BROWN, RAYSMAN, MILLSTEIN, FELDER & STEINER LLP, 900 THIRD AVENUE, NEW YORK, NY, 10022, US

(21) Appl. No.: 11/132,855

(22) Filed: May 18, 2005

Related U.S. Application Data
(60) Provisional application No. 60/639,843, filed on Dec. 27, 2004.

(51) Int. Cl. A61M 31/00

(52) U.S. Cl. 604/500; 604/530; 604/96.01

(57) ABSTRACT

The present invention is directed to a floating anchor, which can be inserted into the esophagus, stomach, small intestine, large intestine, or rectal cavity and reverts to a bent shape when placed therein.

This application claims the benefit of U.S. provisional patent application No. 60/639,843, filed on Dec. 27, 2004, entitled, “Intra-Gastric Anchoring Device For Weight Loss Balloon”, and is incorporated herein by reference.

### BACKGROUND OF INVENTION

1. Field of the Invention

The present invention generally relates to an anchor, which can be placed in the gastro-intestinal tract. Specifically, the present invention is directed to a floating anchor, which can be inserted into the esophagus, stomach, small intestine, large intestine, or rectal cavity and reverts to a bent shape when placed therein. By the placing of the embodiments of the floating anchor as taught by this invention, unwarranted migration of items or devices placed in the gastro-intestinal tract may be safely eliminated.

2. Description of the Related Art

Morbid obesity remains an ever-growing problem in the U.S. Varying forms of gastric bypass surgery have developed and have improved over the last few decades. Recently, laparoscopic gastric banding has emerged as a less invasive surgical option. However, bariatric surgery is fraught with morbidity of up to 20%, with a re-operation rate approaching 25% at 3-5 years post-op. Bariatric surgery carries an operative mortality of 0.5%. Diet and pharmaceutical alternatives have not been very effective, with a high recidivism rate. Today, the Bioenterics™ intragastric balloon (BIB) is in use outside of the U.S., achieving average weight loss of 15 kg and 5 point drop in BMI. However, 8-9% balloon deflation rate has resulted in unwarranted migration leading to obstruction.

There is therefore a great need in the art for an anchor which will not migrate, thus avoiding displacement of whatever device needs to be secured. Accordingly, there is now provided with this invention an intraluminal anchorage method and device effectively overcoming the aforementioned difficulties. These problems have been solved in a convenient and highly effective way by which to insert and anchor balloons, or any other device, in the gastro-intestinal tract.

### SUMMARY OF THE INVENTION

According to one aspect of the invention, a flexible tubular anchor having an elastic memory for assuming a pre-selected bent configuration is described for placement in the gastro-intestinal tract. The anchor comprises a distal end and an open proximal end having a central core extending toward the distal end. When the core receives a straightening rod therethrough, the anchor is straightened from its pre-selected bent shape.

In accordance with another aspect of the invention, a method of inserting a flexible tubular anchor in a patient's gastro-intestinal tract is described. The anchor has an elastic memory for assuming a pre-selected bent shape and has a distal end, an open proximal end having a central core extending toward the distal end, a balloon sealed along a portion of the anchor, an inflation conduit extending from the proximal end to the interior of the balloon, a pushing catheter having a bore therethrough axially aligned with the anchor, and a straightening rod extending through said catheter and the anchor. The method generally comprises inserting the anchor in its straightened configuration into the patient's stomach, separating the anchor from the straightening rod thereby allowing the anchor to assume its pre-selected bent shape, and then inflating the balloon.

As will be appreciated by those persons skilled in the art, a major advantage provided by the present invention is the ease in which an anchor may be inserted into the gastro-intestinal system. Another major advantage provided by the present invention is the safety and security provided by the use of such an anchor. It is therefore an object of the present invention to provide a safe and easy method of inserting and securing a floating anchor into the gastro-intestinal system so that a variety of devices may be safely secured therein. It is another object of the invention to safely and securely anchor a balloon in the stomach for promoting a feeling of satiety in a patient. Additional objects of the present invention will become apparent from the following descriptions.

The method and apparatus of the present invention will be better understood by reference to the following detailed discussion of specific embodiments and the attached figures, which illustrate and exemplify such embodiments.

### DESCRIPTION OF THE DRAWINGS

Specific embodiments of the present invention will be described will reference to the following drawings, wherein:

FIG. 1 is a side view of an embodiment of the present invention.

FIG. 2 is a side view of another embodiment of the present invention.

FIG. 3 is a cross-sectional view of the embodiment depicted in FIG. 2.

FIG. 4 is a side view of the embodiment depicted in FIG. 2, showing an inflated balloon.

FIG. 5 is a side view of another embodiment of the present invention.

FIG. 6 is a side view of the embodiment depicted in FIG. 5, showing an inflated balloon.

FIG. 7 is a side view of an embodiment of the present invention in its pre-inserted configuration.

FIG. 8 is a side view of another embodiment of the present invention, showing an appendage attached to the distal end of the anchor.

FIG. 9 is a side view of another embodiment of the present invention, showing attachments to the wall of the anchor.

FIG. 10 is a side view of another embodiment of the present invention, showing both attachments and a balloon attached to the anchor.

FIG. 11 is a side view of another embodiment of the present invention, showing both a device and a balloon attached to the anchor.

FIG. 12a is a side view of another embodiment of the present invention, showing a helical shaped distal end and a conduit canal within the central core.

FIG. 12b is an enlarged view of the proximal end of the embodiment of FIG. 12a.

### DESCRIPTION OF THE PREFERRED EMBODIMENT

The following preferred embodiment as exemplified by the drawings is illustrative of the invention and is not intended to limit the invention as encompassed by the claims of this invention.

The apparatus 1, as generally illustrated in the figures, is an anchoring device for securing devices in the gastro-intestinal tract. The gastro-intestinal tract, as used herein, includes the esophagus. Although this device will be described in its preferred environment of use for anchoring an inflated balloon in the stomach for affecting weight loss, it is to be understood by persons skilled in the art that the method and device described herein can be used for securing any device for its intended purpose anywhere in the gastrointestinal tract.

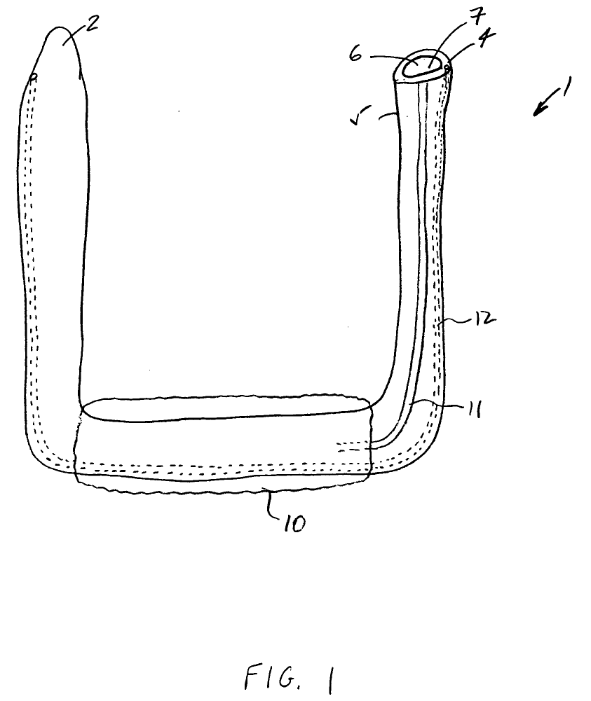

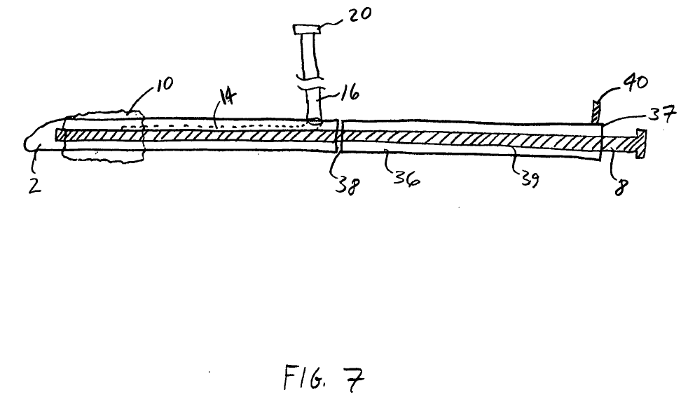

FIG. 1 depicts an embodiment of the floating gastro-intestinal anchor. As shown in the embodiment of FIG. 1, the anchor 1 has a “C” shape. The anchor has a distal end 2, a proximal end 4, and a side wall 5. The distal end is preferentially tapered for ease of insertion. The proximal end 4 has an aperture 6 opening into a central core 7, which extends through substantially the entire length of the anchor. The distal end may be either open or closed. The distal end should preferably be tapered. A rigid insertion rod 8 (shown in FIG. 7 and described below) is inserted into the aperture 7 during insertion of the anchor into the patient. The anchor or catheter 1 is made of a material that is flexible enough to be straightened, but has an elastic “memory” to conform to a pre-selected bent shape. The elastic memory may be imparted by the material itself, or alternatively, by the addition of another material. For example, the shape that the anchor reverts to may be determined by the inclusion of an additional material having a memory such as spring steel or a plastic insert. The anchor material should be made of biocompatible material, preferably radio-opaque, that can withstand the acid milieu of the stomach, as is well known to those skilled in the art. Located approximately midway between the distal end and the proximal end of the anchor can be a balloon 10, which is fixed to and surrounds the anchor. It is shown in FIG. 1 in its deflated state. The balloon should be manufactured of biocompatible material that can withstand the acid milieu of the gastric lumen. A conduit channel 11 can be formed in the sidewall of the anchor. This conduit channel allows for a thin-walled conduit (not shown) to pass along the wall of the anchor and into the interior space of the balloon 10 for eventual inflation thereof. A guide wire canal 12 may preferably be formed in the wall of the anchor 1 for inserting a guide wire during insertion of the anchor into the stomach. Alternatively, as is well known to those skilled in the art, an overtube may be used in lieu of a guide wire during insertion of the anchor into the gastro-intestinal tract. Of course, if the diameter of the anchor is small enough, the biopsy channel of the endoscope itself may be used as an overtube to direct the anchor into the gastro-intestinal tract.

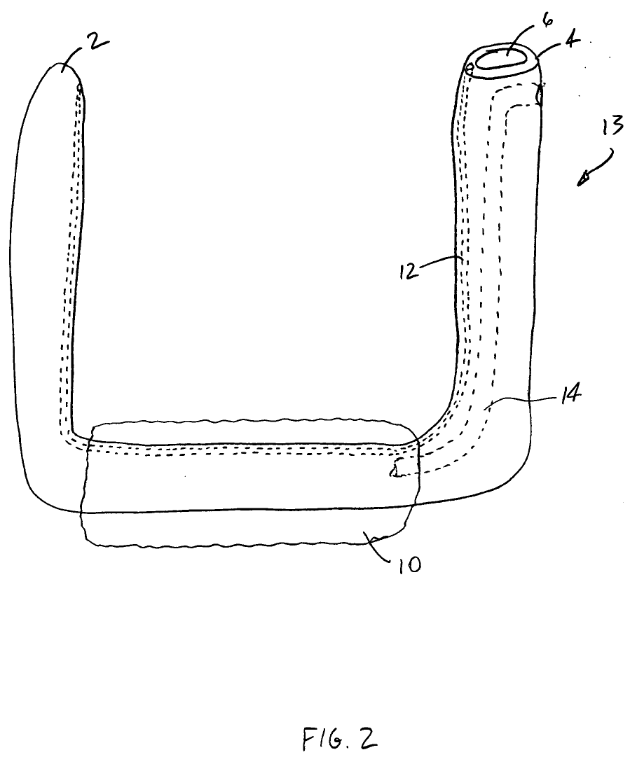

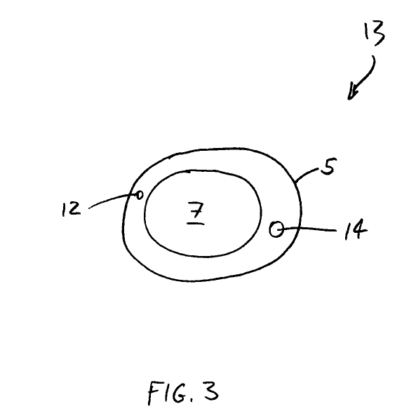

FIGS. 2 and 3 show another embodiment of the anchor 13 of the present invention. In this embodiment, the conduit channel 11 of FIG. 1 has been replaced with a conduit canal 14 for formed within the wall of the anchor 1. Within the wall 5 of the anchor could be a small lumen 12 to allow passage of a standard guide wire, typically 0.28 inches, but of course may vary widely, as is well known in the art (as shown in FIG. 3).

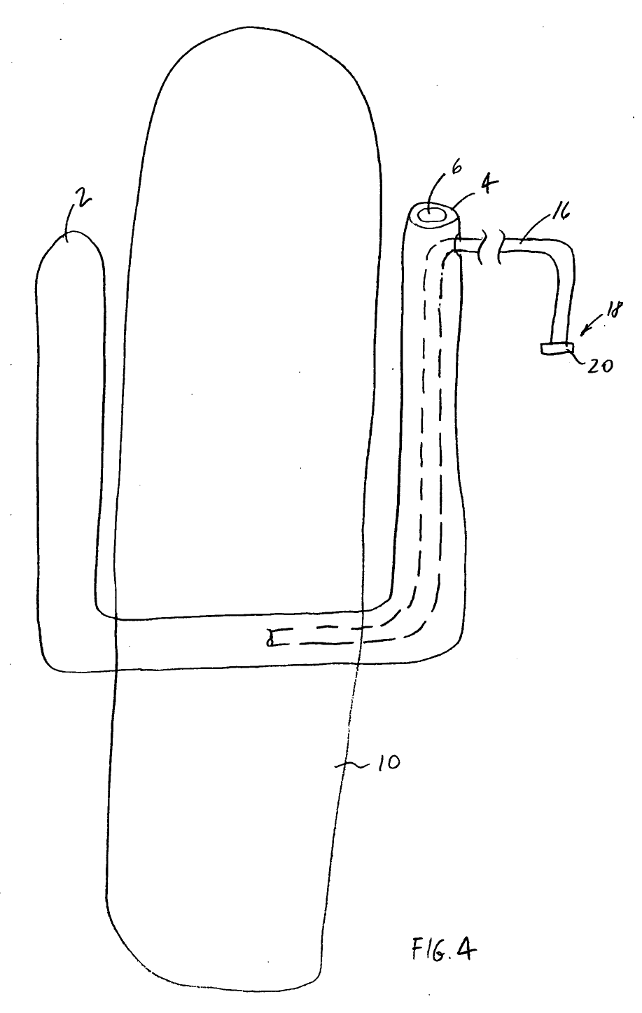

FIG. 4 shows the embodiment of FIG. 2 with a thin walled conduit 16 inserted into the sidewall of the anchor and threaded into the interior of the balloon 10. The balloon 10 is shown in its inflated state in FIG. 4. The proximal end 18 of the conduit 16 will extend a sufficient distance, for example 60 cm, to allow passage out of the mouth while the anchor with the inflated balloon is in the gastric lumen. At the proximal end 18 of the conduit 16 is a fitting 20. This fitting 20 will typically be of a “luer-lock” type or equivalent self-sealing mechanism, as is well known to those skilled in the art, for allowing inflation of a balloon in a sealed system. The insertion of the balloon into the stomach will promote a feeling of satiety in the patient and may interfere with peristaltic waves and gastric emptying.

The caliber of the conduit and its lumen will be sufficient to allow inflation of any balloon on this length conduit. The conduit should be made of a biocompatible material that can withstand the acid milieu, and can flex with the anchor in its different conformations. The balloon(s) will be inflated to the point that they fill the gastric lumen which will be anywhere from 400-1000 cc depending on stomach size. The anchor should preferentially have external markings from the end of the tapered tip every 5 cm for help in guiding the operator.

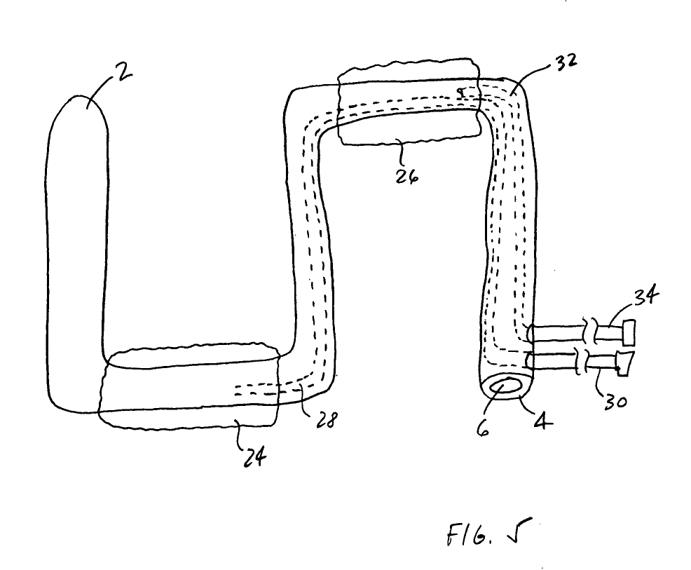

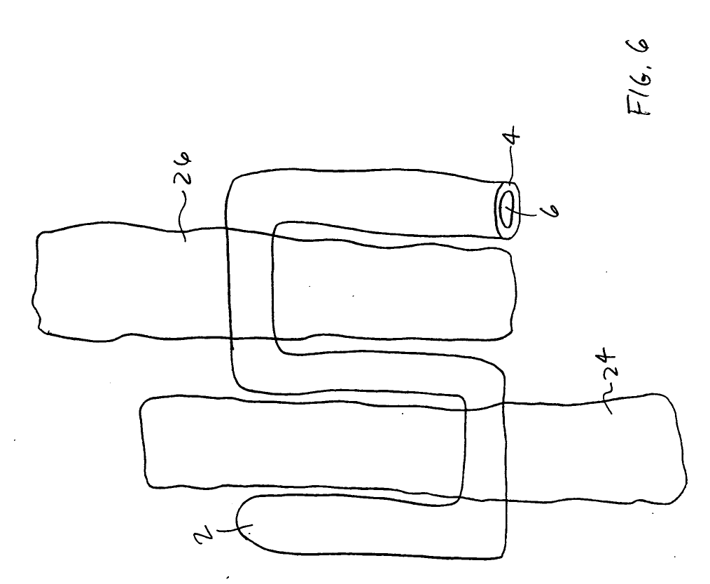

FIG. 5 shows another embodiment of the anchor 22 of the present invention. As illustrated in FIG. 5, the anchor 2 has an “S” configuration with two balloons; a distal balloon 24 and a proximal balloon 26. Alternatively, the length of the anchor proximal to the proximal balloon may not be required. As shown, if more than one balloon is used, then a corresponding number of designated canals are used for guiding conduits into their respective interiors. A distal canal 28 guides distal conduit 30 to the distal balloon 24. A second canal, proximal canal 32 guides proximal conduit 34 to the proximal balloon 26. If two or more balloons are used, then two or more conduits will be used in the same manner. The inflation ports will be marked proximal and distal in such a way that is easily recognizable to an endoscopist upon viewing in the gastric lumen. FIG. 6 shows the embodiment of FIG. 5 with the proximal and distal balloons in their inflated state.

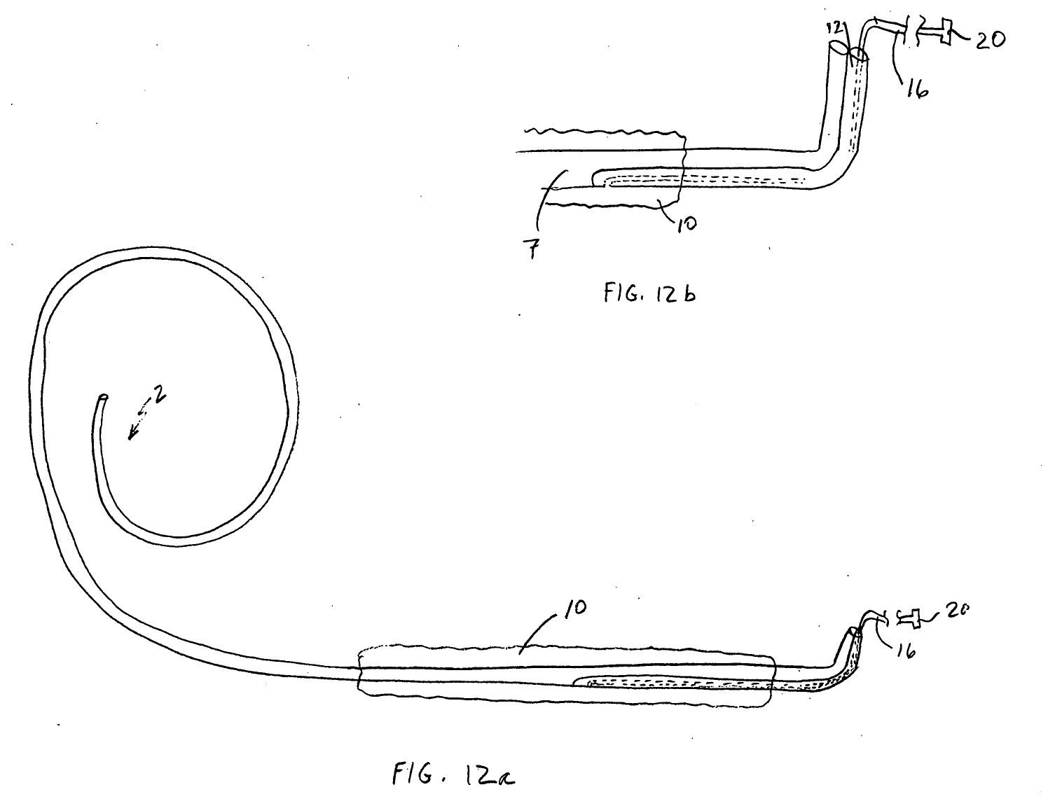

An alternative pre-selected bent shape would be a helical configuration at the distal end of the anchor (FIG. 12). The diameter of the helix would be such that it could not pass through the pylorus, for example, a diameter ranging from about 4 cm to about 20 cm and preferably from about 8cm to about 14cm. On the straight length of the anchor, a therapeutic device, such as a balloon, or a transmitting device, such as a camera or other transmitting device, or other therapeutic device may be attached (FIG. 12). The proximal end may have a curved tip for diminishing tissue trauma.

As shown in FIG. 7, the anchor 1 is in its straightened position prior to insertion. The rigid rod 8 is extended through the entire length of the central core 7. A pushing catheter 36 having a proximal end 37 and a distal end 38 is shown in axial alignment with the anchor 1. The pushing catheter 36 has a bore 39 extending entirely through it. The rod 8 extends entirely through the bore 39 and outside of the proximal end 37 of the pushing catheter. The straightening rod will be made of a rigid unbendable biocompatible material, which easily slips in and out of the catheter. It will be long enough to fully engage the catheter lumen, and extend out beyond another approximately 45 cm. A pushing tab 40 is preferentially placed at the proximal end of the pushing catheter. The pushing catheter will be made of biocompatible rigid unbendable material. It will have at its proximal end a tab that can be grabbed and used to push the anchor off of the rod.

After the anchor has been inserted and the straitening rod has been removed, the anchor will assume its “C”, “S”, “U”, or any other pre-selected bent shape that has been configured into its “memory”. By assuming a pre-selected bent shape, the anchor will prevent migration of the inserted device. The anchor material should be flexible enough to enable straightening for deployment, but will have a memory shape that remains after the straightening or insertion rod 8 is removed. In this way, it will allow safe atraumatic removal endoscopically even with its memory shape, or if needed, may be partially straightened during removal. It will allow carriage of one or more balloons, transmitters, cameras, or any other device that requires its presence in the gastric lumen.

The anchor should preferably be approximately 40 cm long, with each arm approx. 12 cm and the center approximately 16 cm. These dimensions may, of course, vary depending on stomach shape and size. Other areas of the gastro-intestinal tract will require various shapes and sizes. The distal end is closed and should preferentially be tapered with a soft flexible tip to allow easy passage through the gastro-intestinal tract.

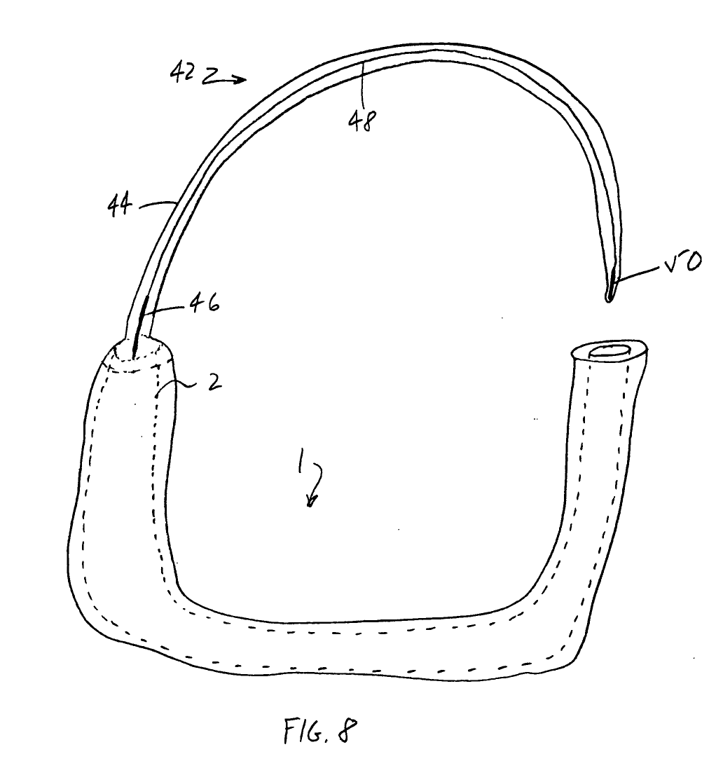

FIG. 8 illustrates a further feature which may be included as part of the anchor of the present invention. Attached to the distal end 2 of the anchor is an appendage 42. The appendage comprises a length of wire that is housed in a flexible shaft 44. The appendage is preferably an elongated continuation of the distal tip of the same material. The appendage is preferably string-like and approximately 3-5 mm in diameter. The length of wire housed therein is preferably a unitary piece preferably comprising a first relatively short segment 46 (approximately 5 cm) which should be relatively flexible. As the wire continues within this “string” appendage, a longer segment of this wire 48 (approximately 8-10 cm) should be relatively stiff, followed by a third flexible segment 50 (approximately 3 cm). This length of wire may alternatively comprise a different arrangement of alternating flexible and stiff segments each having various lengths. When the balloon is inflated, the appendage should preferably push away from the proximal arm so that when the balloon is inflated, this appendage can be easily pushed out of the way. However, when the balloon is deflated, the appendage should preferably assume its position with its distal end approximating the proximal end of the anchor. This appendage 42 offers an added degree of security to the anchor by closing off the gap between the proximal end and the distal end of the anchor thereby preventing any unwarranted migration out of the stomach and into the pylorus.

The proximal end of the anchor will not be tapered, but will be rounded to prevent tissue damage upon contact. The catheter will have approximately 25-35 Fr caliber. The interior surface of the catheter may require a different biocompatible material to allow passage of a straightening rod and/or for shape maintenance. If a separate guide wire canal is not used, the anchor may be of a much smaller caliber of approximately 6 to 16 Fr. As a further alternative, the central core of the anchor may be used for a guide wire or for a guide wire that functions as a straightening rod. As an even further alternative, the straightening rod could have a central core for a guide wire therethrough.

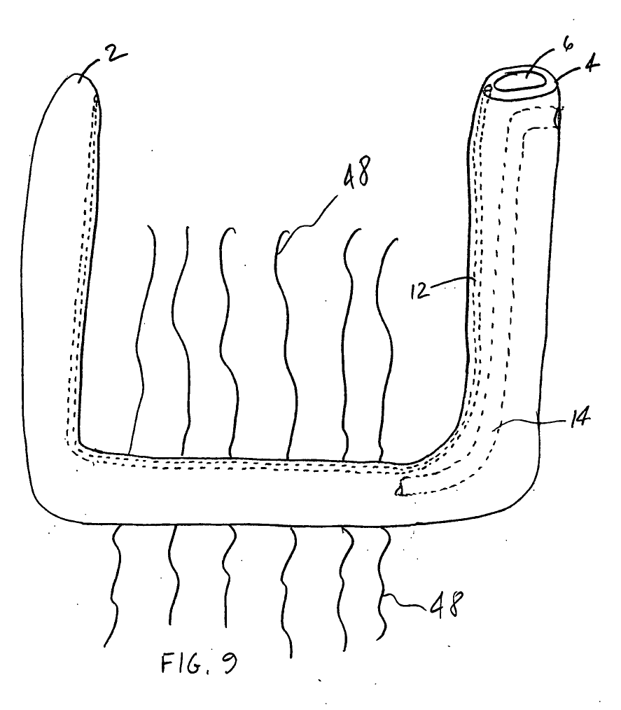

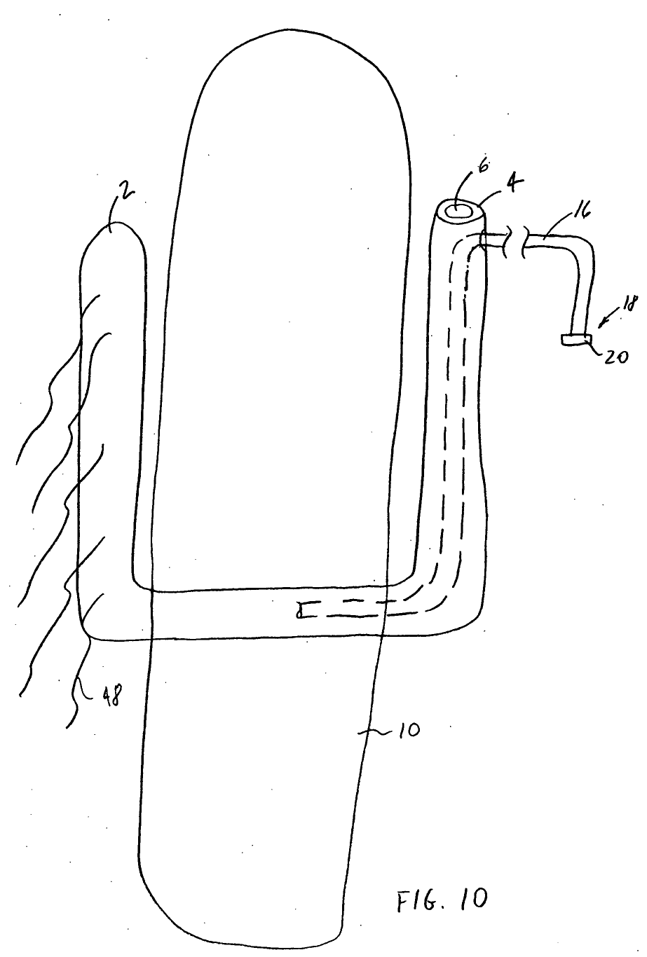

FIG. 9 illustrates another attachment to the anchor. In this figure attachments 48 may be of a wide variety of bio-compatible materials, for example, cord, ribbon, sponges, other thin material, or combinations thereof. The purpose of such attachments is to fill up the antrum and interfering with gastric emptying As shown in FIG. 10 such attachments could be used in combination with the earlier described inflatable balloon.

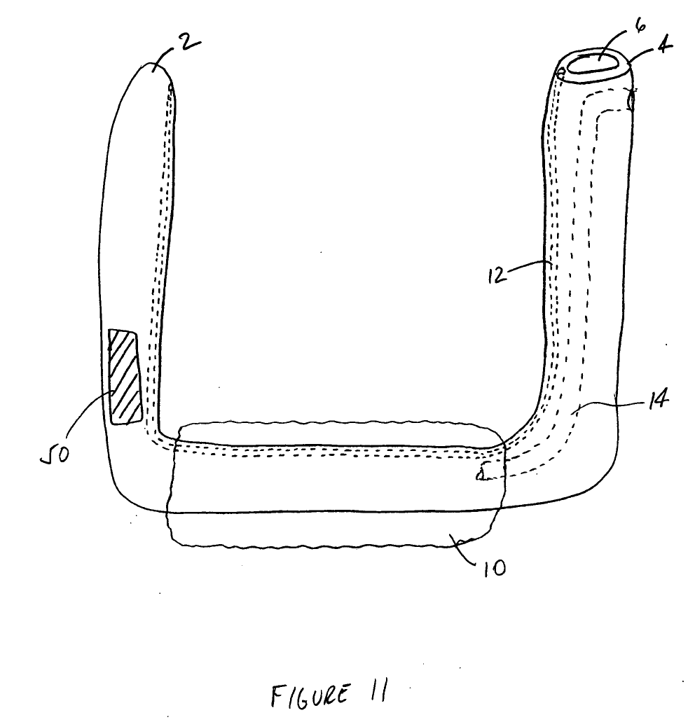

FIG. 11 illustrates a further device 50 connected to the anchor. Such a device may be a transmitting device or any other device, such as a camera which could be placed separately, or in combination with any other device such as the inflatable balloon illustrated in FIG. 11. The additional device 50 depicted in FIG. 11 could also be another type of therapeutic device, for example, it could be a device for administering medication or a device for targeting a tumor. If it would include a device for targeting a tumor, device 50 could include one in which chemotherapy, radiation therapy, photodynamic therapy, tumor ablation therapy, seed implant therapy, or any other therapy known to those skilled in the art is administered.

As shown in FIGS. 12a and 12b, the anchor may assume the bent shape of a helix at its distal end. Further, the conduit canal may be placed within the central core. The balloon may be inflated in the conventional manner.

Deployment

It is to be understood that the following examples of use of the present invention is not intended to restrict the present invention, since many more modifications may be made within the scope of the claims without departing from the spirit thereof.

Deployment can be performed using a gastric overtube or over a guidewire. These are standard well-established techniques. The guidewire method has been described in the upper GI tract in reference to esophageal strictures. Kadakia(1), Fleischer(2) and Dumon(3) have published their results with esophageal dilators passed over guide wires without need for fluoroscopy. In particular, the Savary system guide wire technique (3) would be used with the FGIA catheter deployment. Upper endoscopy is performed with complete evaluation of the esophagus stomach and duodenum. The endoscopist will measure the distance from the incisors to the gastro-esophageal junction. With the endosope in the gastric antrum, the guidewire (flexible tip first) is passed under direct vision into the gastric antrum. The guidewire is advanced as the endoscope is removed leaving the guidewire in the gastric lumen. This has been described with the Savary dilator system (3). The free end of the guide wire (outside of the mouth) is then placed into the guide wire lumen of the anchor. The anchor is slid down over the guidewire (without changing position of the guidewire relative to the mouth) and passed into the mouth down the esophagus. When the external markings on the anchor at the incisors are 6-8 cm greater than the level of the gastro esophageal junction (as noted by the endoscopist during the initial endoscopy), the pushing tab of the pushing catheter is pushed forward while holding the rod in the same position relative to the mouth. Once, the anchor is free of the rod, the rod, guide wire, and pushing catheter are removed. The conduit(s) inflation port(s) should be outside of the mouth. The endoscope is then re-inserted to inspect the position of the anchor and any necessary adjustments are made. The conduit inflation port is then accessed with a luer-lock syringe and inflated approximately 400-1000 cc of the fluid, depending on stomach size, which can be viewed endoscopically. This is repeated with multiple balloons. The conduit tubing is then pulled down into the stomach using a snare, hook catheter, grabbing forceps, or equivalent. Once the conduit tubing is in the gastric lumen, the endoscope is then removed and the procedure is completed. If at a later time the patient needs an adjustment of the balloon(s), endoscopy with snare, hook catheter, grabbing forceps, or equivalent access of the free end of the conduit tubing can be done. The conduit is pulled out of the mouth and inflation or deflation performed, followed by pulling the free end of the conduit into the gastric lumen as described above. As is well known to those skilled in the art, the many embodiments described herein may be placed into the gastro-intestinal tract by many methods of insertion. (1) Kadakia SC et al. Esophageal dilation with polyvinyl bougies using a marked guidewire without the aid of fluoroscopy. Am J Gastro 1993;88:1381-86; (2) Fleischer DE et al. A marked guidewire facilitates esophageal dilation. Am J Gastro 1989;84:359-61; (3) Dumon J R Et Al. A new method of esophageal dilation using Savary-Gilliard bougies. Gastro Endosc 1985:31:379-82.

If the gastric overtube method is used, again standard endoscopy is performed with inspection of the esophagus stomach and duodenum. The endoscope is then removed and a gastric overtube is placed, as per usual technique. Werth et al. (4) described use of a gastric overtube for foreign body removal and for multiple endoscopic intubations. Once the overtube is in place, the anchor is placed through the overtube. As described above with the guidewire technique, the pushing catheter tab is pushed once the distal tip of the anchor is 6-8 cm beyond the gastro esophageal junction. When the anchor is off the rod, the rod and pushing catheter are removed. The endoscope can then pass through the overtube and inspect the anchor position as mentioned above. The remainder of the procedure is the same. The anchor, having reverted to its pre-selected bent shape, will prevent a deflated balloon from migrating, leading to a safer weight loss device. The anchor can be used to anchor more than one balloon, and can anchor any device that needs to remain in the gastric lumen. It can be used in a post bariatric surgical patient whose weight loss has plateaued and wishes further weight loss. It can be used anywhere in the GI tract where a device of any kind needs to remain in place. The site of the anchor will determine its pre-determined shape, geometry, and size. It is placed endoscopically and removed endoscopically under conscious sedation in an outpatient setting.

Although the particular embodiments shown and described above will prove to be useful in many applications in the endoscopic art to which the present invention pertains, further modifications of the present invention will occur to persons skilled in the art. All such modifications are deemed to be within the scope and spirit of the present invention, as defined by the appended claims.

What is claimed is:

1. A flexible tubular anchor having an elastic memory for assuming a pre-selected bent configuration, for placement in the gastro-intestinal tract, comprising:
a) a therapeutic device attached to the anchor;
b) a distal end and an open proximal end having a central core extending toward said distal end; and
wherein, when said core receives a straightening rod therethrough, the anchor is straightened from its pre-selected bent shape.

2. The anchor of claim 1, wherein said therapeutic device includes a balloon sealed along a portion of the anchor, and wherein the anchor further comprises a conduit extending from said proximal end to the interior of the balloon for inflation thereof.

3. The anchor of claim 2, wherein said distal end is tapered.

4. The anchor of claim 3, wherein the bent shape is selected from the group comprising: “C”, “S”, “U”, helical, sinusoidal, or any other migration precluding shape.

5. The anchor of claim 4, further comprising a second balloon and a second conduit extending to the interior of said second balloon for inflation thereof.

6. The anchor of claim 5, further comprising a pushing catheter having a bore therethrough, axially aligned with the anchor and having a straightening rod extending through said catheter and the anchor.

7. The anchor of claim 1, further comprising an elongated appendage extending from said distal end comprising a housing having a wire therein, wherein said wire comprises a first flexible segment, a second relatively stiff segment, and a third relatively flexible segment.

8. The anchor of claim 1, further comprising a guide wire canal in the anchor wall.

9. The anchor of claim 1, further comprising a floating attachment for interfering with gastric emptying.

10. The anchor of claim 1, further comprising a transmitting device.

11. The anchor of claim 1, wherein said therapeutic device includes string-like attachments.

12. The anchor of claim 1, wherein said therapeutic device is for administering medication.

13. The anchor of claim 1, wherein said therapeutic device is for administering tumor targeting therapy.

14. A flexible tubular anchor having an elastic memory for assuming a pre-selected bent configuration, for placement in the gastro-intestinal tract, comprising:
a) a transmitting device attached to the anchor;
b) a distal end and an open proximal end having a central core extending toward said distal end; and
wherein, when said core receives a straightening rod therethrough, the anchor is straightened from its pre-selected bent shape.

15. The anchor of claim 14, wherein said distal end is tapered.

16. The anchor of claim 15, wherein the bent shape is selected from the group comprising: “C”, “S”, “U”, helical, sinusoidal, or any other migration precluding shape.

17. The anchor of claim 14, further comprising a pushing catheter having a bore therethrough, axially aligned with the anchor and having a straightening rod extending through said catheter and the anchor.

18. The anchor of claim 14, further comprising an elongated appendage extending from said distal end comprising a housing having a wire therein, wherein said wire comprises a first flexible segment, a second relatively stiff segment, and a third relatively flexible segment.

19. The anchor of claim 14, further comprising a guide wire canal in the anchor wall.

20. The anchor of claim 14, further comprising a floating attachment for interfering with gastric emptying.

21. The anchor of claim 14, further comprising string-like attachments.

22. The anchor of claim 14, further comprising a therapeutic device for administering medication.

23. The anchor of claim 14, further comprising a therapeutic device for administering tumor targeting therapy.

24. A flexible tubular anchor having an elastic memory for assuming a pre-selected bent configuration, for placement in the gastro-intestinal tract, comprising:
a) a closed distal end;
b) an open proximal end having a central core extending toward said distal end; and
wherein, when said core receives a straightening rod therethrough, the anchor is straightened from its pre-selected bent shape.

25. The anchor of claim 24, further comprising:
a) a balloon sealed along a portion of the anchor; and
b) a conduit extending from said proximal end to the interior of the balloon for inflation thereof.

26. The anchor of claim 25, wherein said distal end is tapered.

27. The anchor of claim 26, wherein the bent shape is selected from the group comprising: “C”, “S”, “U”, helical, sinusoidal, or any other migration precluding shape.

28. The anchor of claim 27, further comprising a second balloon and a second conduit extending to the interior of said second balloon for inflation thereof.

29. The anchor of claim 26, further comprising a pushing catheter having a bore therethrough, axially aligned with the anchor and having a straightening rod extending through said catheter and the anchor.

30. The anchor of claim 24, further comprising an elongated appendage extending from said distal end comprising a housing having a wire therein, wherein said wire comprises a first flexible segment, a second relatively stiff segment, and a third relatively flexible segment.

31. The anchor of claim 24, further comprising a guide wire canal in the anchor wall.

32. The anchor of claim 24, further comprising a floating attachment for interfering with gastric emptying.

33. The anchor of claim 24, further comprising string-like attachments.

34. The anchor of claim 24, further comprising a therapeutic device for administering medication.

35. The anchor of claim 24, further comprising a therapeutic device for administering tumor targeting therapy.

36. A method of inserting a flexible tubular anchor in a patient's gastrointestinal tract, wherein said anchor has an elastic memory for assuming a pre-selected bent shape and has a distal end; an open proximal end having a central core extending toward said distal end; a balloon sealed along a portion of the anchor; an inflation conduit extending from said proximal end to the interior of the balloon; a pushing catheter having a bore therethrough axially aligned with the anchor; and a straightening rod extending through said catheter and the anchor; comprising:
a) inserting the anchor in its straightened configuration into the patient's gastro-intestinal tract;
b) separating the anchor from the straightening rod thereby allowing the anchor to assume said pre-selected bent shape; and
c) inflating the balloon.

37. The method of claim 36, wherein said insertion step includes threading the anchor onto a guide wire.

38. The method of claim 36, wherein said insertion step includes inserting the anchor into an overtube.

39. The method of claim 36, wherein said insertion step includes pushing the anchor off the rod by pushing on the pushing catheter.

40. The method of claim 36, further comprising inserting the inflation conduit into the patient's stomach.

41. A method of inserting a flexible tubular anchor in a patient's gastro-intestinal tract, wherein said anchor has an elastic memory for assuming a pre-selected bent shape and has a distal end; an open proximal end having a central core extending toward said distal end; a device attached to the anchor; a pushing catheter having a bore therethrough axially aligned with the anchor; and a straightening rod extending through said catheter and the anchor; comprising:
a) inserting the anchor in its straightened configuration into the patient's gastro-intestinal tract; and
b) separating the anchor from the straightening rod thereby allowing the anchor to assume said pre-selected bent shape.

42. The method of claim 41, wherein said insertion step includes threading the anchor onto a guide wire.

43. The method of claim 41, wherein said insertion step includes inserting the anchor into an overtube.

44. The method of claim 41, wherein said insertion step includes pushing the anchor off the rod by pushing on the pushing catheter.

45. A method of inserting a flexible tubular anchor in a patient's gastro-intestinal tract, wherein said anchor has an elastic memory for assuming a pre-selected bent shape and has a closed distal end; an open proximal end having a central core extending toward said distal end; a pushing catheter having a bore therethrough axially aligned with the anchor; and a straightening rod extending through said catheter and the anchor; comprising:
a) inserting the anchor in its straightened configuration into the patient's gastro-intestinal tract; and
b) separating the anchor from the straightening rod thereby allowing the anchor to assume said pre-selected bent shape.

46. The method of claim 45, wherein said insertion step includes threading the anchor onto a guide wire.

47. The method of claim 45, wherein said insertion step includes inserting the anchor into an overtube.

48. The method of claim 45, wherein said insertion step includes pushing the anchor off the rod by pushing on the pushing catheter.
